(19) United States
(12) Patent Application Publication
(10) Pub. No.: US 2018/0044818 A1
(43) Pub. Date: Feb. 15, 2018

(54) HIGH-AMYLOSE STARCH- FORMATE ELECTROSPUN FIBERS

(71) Applicant: NanoSpun Technologies Ltd., Yokneam (IL)

(72) Inventors: Eyal ZUSSMAN, Haifa (IL); Anica LANCUSKI, Haifa (IL)

(21) Appl. No.: 15/552,489

(22) PCT Filed: Feb. 22, 2016
(86) PCT No.: PCT/IL2016/050201

§ 371 Date: Aug. 21, 2017

Related U.S. Application Data
(60) Provisional application No. 62/119,197, filed on Feb. 22, 2015.

(51) Int. Cl. D01F 8/18; A61K 9/00; D01F 9/00; D01D 7/00; A61K 35/747; D01D 5/00; D01D 4/02; D01D 5/38; A61K 9/70; D01F 1/10

(52) U.S. Cl. CPC D01F 8/18; A61K 9/70; A61K 9/0053; D01F 9/00; D01F 1/10; A61K 35/747; D01D 5/0038; D01D 4/02; D01D 5/38; D01D 7/00; D10B 2509/00; D10B 2403/024

(57) ABSTRACT

Starch-based fibers, compositions comprising same, method of preparing said starch-based fibers, kits and methods for use of said starch-based fibers including but not limited to oral delivery of cells (e.g., probiotic microorganisms) and/or molecules of interest (e.g., nutrients) are provided.

### REFERENCE TO RELATED APPLICATION

This application claims priority from U.S. Provisional Patent Application Ser. No. 62/119,197, filed Feb. 22, 2015, the entire content of which is incorporated herein by reference.

### FIELD OF INVENTION

The present invention is directed to, inter alia, electrospun high-amylose starch-formate fibers.

### BACKGROUND OF THE INVENTION

There is a constant and progressive trend of replacement of synthetic polymers from limited petrol-based resources with sustainable natural macromolecules and their derivatives. Starch is natural, abundant polysaccharide present mainly in plants as energy storage. As FDA-approved, GRAS (generally recognized as safe), and biocompatible material, starch has been intensively used in medical, pharmaceutical and food industries as an inexpensive support for drug delivery and food packaging. However, pure starch materials, typically starch films, are rather brittle, water-sensitive and difficult to be processed, thus with limited applications.

In order to overcome the brittleness of starch films, patents such as U.S. Pat. No. 4,853,168 and U.S. Pat. No. 6,709,526 among others, relate their innovation towards processing starch fibers, such as by extrusion- or melt-spinning with a help of additives to improve the flow and processability. Additionally, the orientation of the starch in the fibers was reported by Wolff, I. (Ind. Eng. Chem. 1958, 50, 1552-1552) to improve mechanical properties of the material, typically tensile strength and elongation at break. Kong and Ziegler (Recent Pat. Food Nutr. Agric. 2012, 4, 210-219; Biomacromolecules 2012, 13, 2247-2253; and Carbohydr. Polym. 2013, 92, 1416-1422) studied processing of pure starch fibers with electrospinning technique from DMSO-rich solvent medium and proposed quantitative relationships between electrospinning parameters and fiber diameter (Doshi, J.; Reneker, D. H. J. Electrost. 1995, 35, 151-160; Reneker, D. H.; Chun, I. Nanotechnology 1996, 7, 216; Theron, S. A.; Zussman, E.; Yarin, A. L. Polymer 2004, 45, 2017-2030; Rungswang, W et al. Polymer 2011, 52, 844-853). However, due to the poor mechanical properties they observed upon the fibers' handling, and in order to improve the crystallinity and water-stability of the fibers, these authors turned toward post-processing treatments of annealing and crosslinking (Kong, L.; Ziegler, G. R. Food Hydrocoll. 2014, 36, 20-25; and Kong, L.; 219), as well as formation of starch-guest inclusion complexes (Kong, L.; Ziegler, G. R. Carbohydr. Polym. 2014, 111, 256-263; Ziegler, G. R. Food Hydrocoll. 2014, 38, 211-). In order to improve mechanical properties of the final starch-based product but to avoid synthetic additives, Xu et al. proposed to chemically modify the starch prior electrospinning (Biotechnol. Prog. 2009, 25, 1788-1795). After the synthesis of starch-acetate from high-amylose maize starch and acetic anhydride, the purified product was electrospun into fibers from formic acid (Xu, Y. et al. Cereal Chem. J. 2004, 81, 735-740).

It is well documented that the starch in formic acid (FA) undergoes rapid esterification, called o-formylation. The action of FA on starch at ambient temperatures is regioselective, giving mono-formate esters at C6 position of glucose units of starch, and has a reversible character reaching equilibrium after ˜8 hours in 90% formic acid solution. Reversibility of the acetylation reaction is used in preparation of orientated starch films. O-formylation of starch is also known as one of the methods to degrade the initial granule structure of the starch and enable better mixing properties with other biodegradable polymers. Formic acid is also a chemical product from y irradiation of maize starch and it represents the main part of radio-induced free acidity. These examples highlight interesting, versatile, yet briefly studied properties starch/FA system.

### SUMMARY OF THE INVENTION

The present invention provides, in some embodiments, starch-based fibers, compositions comprising same, method of preparing said starch-based fibers, kits and methods for use of said starch-based fibers including but not limited to oral delivery of cells (e.g., probiotic microorganisms) and/or molecules of interest (e.g., nutrients).

According to one aspect, there is provided a method of making a starch-formate fiber, the method comprising the steps of: providing a first spinning dope comprising a solution or dispersion of starch in a solvent comprising at least 50% vol. formic acid, where the starch is present at a concentration above the critical entanglement concentration where starch fibers are to be produced; and electrospinning the spinning dope to produce a starch-formate fiber.

According to another aspect, there is provided a method of making a starch-formate concentric multi-layered fiber, the method comprising the steps of: providing a first spinning dope for forming at least one layer of the fiber, the first spinning dope comprises a solution or dispersion of starch in a solvent comprising at least 50% vol. formic acid; providing one or more additional spinning dopes for forming at least one additional layer within said fiber; and co-electrospinning the spinning dopes through multi-axial capillaries to produce a starch-formate concentric multi-layered fiber. In one embodiment, the starch is present at a concentration above the critical entanglement concentration where starch fibers are to be produced.

According to some embodiments, said solvent of the first spinning dope comprises at least 50% vol. formic acid. According to some embodiments, said solvent of the first spinning dope comprises at least 90% vol. formic acid. According to some embodiments, said solvent of the first spinning dope comprises less than 30 vol. % water. According to some embodiments, said solvent comprises 70-100 vol. % formic acid and 0-30 vol. % water.

According to some embodiments, said first spinning dope comprises an aqueous solution or dispersion of starch in a solvent.

According to some embodiments, said first spinning dope comprises 5-40 wt. % starch. According to some embodiments, said starch is high-amylose starch. According to some embodiments, said starch has an amylose:amylopectin ratio of 60:40-95:5.

According to some embodiments, said first spinning dope comprises 10-30 wt. % starch in 70-100 vol. % formic acid.

According to some embodiments, the method of the invention (e.g., the electrospinning step) takes place in a temperature in the range of 18-24° C.

According to some embodiments, the first spinning dope is for forming a shell and the additional spinning dope is for forming a coat over an internal surface of said shell.

According to some embodiments, said one or more of said spinning dopes comprises cells and/or molecules of interest. According to some embodiments, said cell is an animal cell. According to some embodiments, said cell is a probiotic microorganism. According to some embodiments, said probiotic microorganism is selected from the group consisting of bacteria, yeast and mold, or combinations thereof.

According to another aspect, the present invention provides a fiber comprising electrospun starch-formate, said starch has an amylose:amylopectin ratio of 60:40-95:5.

According to another aspect, the present invention provides a concentric multi-layered fiber comprising at least one layer comprising starch-formate, said starch has an amylose:amylopectin ratio of 60:40-95:5.

According to another aspect, there is provided a composition comprising the fiber of the invention. According to some embodiments, said composition comprising the fiber of the invention is for oral administration of viable and physiologically active microorganisms and/or at least one compound of interest to an individual in need thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D are a schematic illustration of the action of FA on starch in pure FA: A) complete granule-destructuration and network formation of starch-formate and B) conformational evolution during electrospinning of the starch-formate network, and in aqueous FA: C) partial granule swelling, aggregation and new type network formation of starch-formate and unreacted aggregates and D) their possible conformational evolution during the electrospinning process.

FIG. 2. FTIR-ATR spectra of HYLON VII® starch film cast from: DMSO (solid line) and formic acid (dotted line).

FIG. 3. Plot of particles' size distribution by volume as a function of the water content (vol. %) in FA/water solvent mixture.

FIG. 4. Viscosity in function of time for 17 wt. % solution of starch-formate in: pure (triangles), 90 vol. % (squares) and 80 vol. % formic acid solution (circles). Blue-shaded area represents optimal rheological properties of the solution for the purposes of electrospinning.

FIG. 5. Viscosity η (depicted under the filled squares and circles) and complex viscosity |η*| (depicted under the open squares and circles) as a function of water content for 17 wt. % starch at different shear rates/frequencies applied: 0.1 s⁻¹ (squares) and 100 s⁻¹ (circles) after 120 h of dissolution.

FIGS. 6A-C. Frequency sweep measurements made on 17 wt. % starch in FA with different water content and at different periods of time: after 1 day (6A), 2 days (6B) and 4 days (6C). 0.4 rad/s is depicted under the filled/open square dotted line 40 rad/s is depicted under the filled/open circle dotted line.

FIG. 7. SEM images of electrospun starch-formate fibers from 24 hours old solutions of 17 wt. % of HYLON® VII starch dissolved in: (A) pure (HS17-pFA), (B) 90 vol. % (HS17-FA90), and (C) 80 vol. % formic acid (HS17-FA80).

FIG. 8. WAXS patterns of (A) dry starch-formate fibers and HYLON® VII powder, (B) dry oriented and isotropic electrospun fibers HS17-FA80 and (C) oriented electrospun (hydrated and dry) fibers HS17-pFA in a capillary.

FIG. 9. Typical strain (σ)-stress (ε) curves of starch-formate electrospun fibers electrospun from: pure formic acid HS17-pFA (squares), 90 vol. % formic acid HS17-FA90 (circles), and 80 vol. % formic acid HS17-FA80 (diamonds). For better visibility of the graphic, each 4^{th} point was presented.

FIGS. 10A-C. Flow curves of Hylon VII starch in 80 vol. % formic acid at different starch concentrations (wt. %) at 25° C. (10A); Flow curves of Hylon VII starch in 90 vol. % formic acid at different starch concentrations (wt. %) at 25° C. (10B); Flow curves of Hylon VII starch in pure formic acid at different starch concentrations (wt. %) at 25° C. (10C).

### DETAILED DESCRIPTION OF THE INVENTION

The present invention demonstrates for the first time a straight-forward method for processing starch from low molecular weight organic acids and particularly solutions comprising formic acid (FA). Without wishing to be bound by any theory, formic acid plays a dual role in simultaneous destruction of the starch granule-structure, esterification of starch to starch-formate and as dispersing solvent for electrospinning process. As demonstrated hereinbelow, at ambient temperatures during the solution preparation and electrospinning process, nanofibers with the diameters of about 200 nm were produced. Rheological measurements evidenced complete starch-granule destruction in pure formic acid solutions at ambient temperatures, while the destruction was only partial for aqueous dispersions of starch-formate in formic acid. Final fibrous mat showed decreased crystallinity and improved mechanical properties thereby exhibiting an economic and ecological biomaterial, for use in many industries, including but not limited to, food packaging, nutraceutical and pharmaceutical industry.

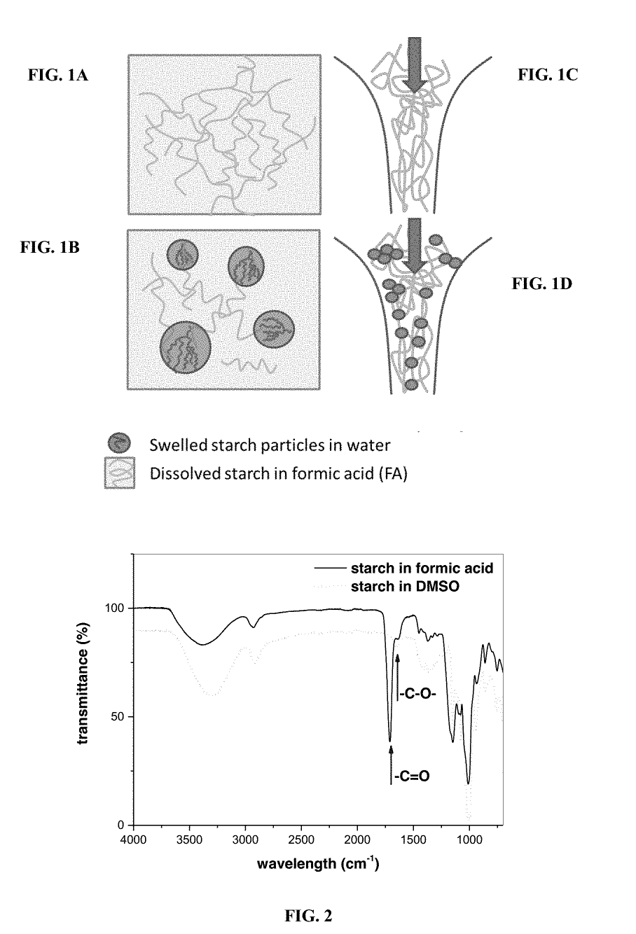

As demonstrated hereinbelow, starch gelatinization mechanism was studied, namely the process of breaking down the intermolecular bonds of starch molecules in pure and diluted FA dispersions and their ability to form fibers via electrospinning. Gelatinization mechanism of starch in water is a well-known and exhaustively studied phenomenon. In semi-diluted and concentrated aqueous dispersions (10 to 30 wt. %), and above the gelation temperature, starch undergoes a simultaneous swelling of granules, melting of crystallites, segregation of amylose from amylopectin and disentanglement of double helices. These complex changes in macromolecular organization are strongly dependent on the temperature (quenching temperature, heating and cooling rate), water content, and the ratio of amylose to amylopectin in the starch as well as the differences in solubility of these polymers. Conversely, when formic acid is used as a solvent, natural wheat starch showed starch granule swelling and decrease in crystallinity even at ambient temperatures. Pure FA induced a complete destructuration of granule and random-coil conformation of starch-formate (FIG. 1A). Thus, the present inventors sought to utilize and examine whether this system, if exceeding the critical entanglement concentration, can form fibers under high electric field (FIG. 1B). While pure formic acid is capable to completely destructurate the starch granule and its crystallinity at room temperature (RT), when 40% FA was used, gelation temperature of the starch significantly increased and only partial swelling and starch aggregation was noted at RT (FIG. 1C). It was suggested that the origin of aggregation might be due to the amphiphilic character of partially o-formylated amylopectin. In this kind of system, the physical entanglement of dissolved fraction of starch (consisted mainly of formylated amylose) would decrease, inducing the decrease in solution viscoelasticity. Viscoelasticity is known in the art as a crucial parameter for electrospinning of continuous fibers. As demonstrated hereinbelow, it is possible to form fibers from concentrated dispersions of starch from aqueous formic acid solutions (FIG. 1D). Further, unlike typical brittle starch films, starch-formate nanofibers demonstrated higher flexibility and elongation at break (6=26%), with the nanometer-size diameters (80-300 nm).

Thus, the present invention provides starch-based electrospun fibers, such as high-amylose starch electrospun fibers, compositions comprising same and processes for producing same. The present invention further provides methods of using the starch-based fibers of the invention in various applications including, but not limited to, wound dressings, drug delivery and sustained release, filtration, and in other areas of the food, cosmetics, textile, and medical and biomedical industries.

The term “fiber”, as used herein, refers to an elongated structure which has a length at least 100 times its width or diameter. Microfibers and nanofibers are produced by methods of the present invention having micro- and/or nanoscale dimensions of length and width or diameter. A cross section of a fiber may have any shape but is typically a circle or oval. The starch fibers and starch particles have a diameter in the range of 1-999 nanometers according to aspects of the present invention. The starch fibers and starch particles have a diameter in the range of 1-999 micrometers according to aspects of the present invention.

According to some embodiments, there is provided a fiber comprising electrospun starch-formate, said starch has a high-amylose content. According to some embodiments, there is provided a concentric multi-layered fiber comprising at least one layer comprising starch-formate, said starch has an amylose:amylopectin ratio of 60:40-95:5. According to some embodiments, there is provided a composition comprising the fiber of the invention and a carrier.

According to some embodiments, there is provided a method of making a starch fiber, the method comprising the steps of: providing a first spinning dope comprising an solution or dispersion of starch in a solvent, where the starch is present at a concentration above the critical entanglement concentration where starch fibers are to be produced; electrospinning the spinning dope to produce a starch fiber.

According to some embodiments, there is provided a method of making a starch-based concentric multi-layered fiber, the method comprising the steps of: providing a first spinning dope for forming at least one layer of the fiber, the first spinning dope comprises a solution or dispersion of starch in a solvent, where the starch is present at a concentration above the critical entanglement concentration where starch fibers are to be produced; providing one or more additional spinning dopes for forming at least one additional layer within said fiber; and co-electrospinning the spinning dopes through multi-axial capillaries to produce a starch-based concentric multi-layered fiber.

According to some embodiments, starch having a high-amylose content has at least 50% wt. %, at least 55% wt. %, at least 60% wt. %, at least 65% wt. %, at least 70% wt. %, at least 75% wt. % or at least 80% wt. % amylose, wherein each possibility represent a separate embodiment of the present invention.

According to some embodiments, starch having a high-amylose content has an amylose:amylopectin ratio of 60:40-95:5. According to some embodiments, starch having a high-amylose content has an amylose:amylopectin ratio of 65:35-95:5. According to some embodiments, starch having a high-amylose content has an amylose:amylopectin ratio of 70:30-95:5. According to some embodiments, starch having a high-amylose content has an amylose:amylopectin ratio of 75:25-95:5. According to some embodiments, starch having a high-amylose content has an amylose:amylopectin ratio of 80:20-95:5.

Starches included in methods and starch fiber compositions according to aspects of the present invention can be any naturally occurring starch, synthetic and/or physically or chemically modified starch.

In some embodiments, said starch is resistant starch (RS). As used herein “resistant starch” refers to starch which resist digestion in the human body such as in the small intestine. Resistant starch is typically categorized into four types: RS1 is a physically inaccessible or digestible resistant starch, such as that found in seeds or legumes and unprocessed whole grains; RS2 is a resistant starch that occurs in its natural granular form, such as uncooked potato, green banana and high amylose corn; RS3 is a resistant starch that is formed when starch-containing foods are cooked and cooled such as in legumes, bread, cornflakes and cooked-and-chilled potatoes, pasta salad or sushi rice; and RS4 is starches that have been chemically modified to resist digestion. In some embodiments, said starch is selected from RS1, RS2, RS3, RS4 and combinations thereof.

According to embodiments relating to multi-layered fibers, two or more types of starch may be used. According to said embodiments, at least one layer comprises starch-formate and at least one additional layer may be formed by a second starch selected from naturally occurring starches, synthetic starches, and/or physically or chemically modified starches of various amylose content, including, but not limited to, starch acetate, starch phosphates, starch succinates, hydroxypropylated starches, dextrin roasted starches, acid treated starches, alkaline treated starches, oxidized starches, bleached starches, enzyme-treated starches, examples of which include, but are not limited to acetylated distarch adipate, acetylated oxidized starch, monostarch phosphate, distarch phosphate, phosphated distarch phosphate, acetylated distarch phosphate, hydroxypropyl starch, hydroxypropyl distarch phosphate and starch sodium octenylsuccinate. According to one embodiment, said chemically modified starch is other than starch acetate.

A sufficient amount of starch is dissolved or dispersed in a solvent or dispersant so that the starch concentration is above its critical entanglement concentration (cₑ). To determine the critical entanglement concentration, specific viscosity data may be plotted versus concentration on a log-log plot. Specific viscosity is where go is zero shear rate viscosity and ηₛ is the solvent viscosity.

ηₛₚ=(η₀−ηₛ)/ηₛ,

The zero shear rate viscosity can be estimated, using the actual or extrapolated values for apparent viscosity at 0.1 s⁻¹, for example. The critical entanglement concentration cₑ is defined as the concentration at which a slope change is observed at the crossover between the semidilute unentangled regime and the semidilute entangled regime of a polymer solution. In the semidilute unentangled regime, polymer chains overlap one another but do not entangle, whereas in the semidilute entangled regime, polymer chains significantly overlap one another such that individual chain motion is constrained.

In some embodiments, starch-based fiber compositions provided according to aspects of the present invention include at least 50, 60, 70, 80, 90, 95, 99 or greater wt. % starch.

The term “spinning dope” as used herein refers to a composition subjected to wet-electro spinning or wet-electrospraying according to methods of the present invention.

According to some embodiments, said solvent of the first spinning dope is one or more low molecular weight organic acid. According to some embodiments, said low molecular weight organic acid is selected from the group consisting of: formic acid, acetic acid, propionic acid, butyric acid, isobutyric acid, caproic acid, lactic acid, oxalic acid, fumaric acid, maleic acid, malonic acid, succinic acid and combinations thereof.

According to some embodiments, said first spinning dope further comprises a denaturing agent. Non-limiting examples of denaturing agents include alcohol such as methanol (CH3OH) or ethanol (CH3CH2OH), or a fluorinated alcohol such as 3,3,3,3′,3′,3′-hexafluoro-2-propanol (HFIP; (CF3)2CHOH) or 2,2,2-Trifluoroethanol (TFE; CF3CH2OH). According to some embodiments, said solvent of the first spinning dope comprises an alcohol based solvent. According to some embodiments, said alcohol is selected from the group consisting of: methanol, ethanol, 1-propanol, isopropyl alcohol, butyl alcohol, amyl alcohol, pentanol, hexanol, heptanol; and a mixture of any two or more thereof.

According to some embodiments, said solvent of the first spinning dope is formic acid. According to some embodiments, said solvent of the first spinning dope is formic acid. A combination of formic acid and at least one additional solvent. According to some embodiments, said solvent of the first spinning dope comprises at least 50% vol., at least 55% vol., at least 60% vol., at least 65% vol., at least 75% vol., at least 80% vol., at least 85% vol., at least 90% vol., at least 95% vol., or at least 99% vol. formic acid. According to some embodiments, said solvent of the first spinning dope comprises at least 90% vol. formic acid. According to some embodiments, said solvent of the first spinning dope is an aqueous formic acid solution.

According to some embodiments, said solvent of the first spinning dope comprises less than 30 vol. % water. According to some embodiments, said solvent of the first spinning dope comprises less than 25 vol. % water. According to some embodiments, said solvent of the first spinning dope comprises less than 20 vol. % water. According to some embodiments, said solvent of the first spinning dope comprises less than 15 vol. % water. According to some embodiments, said solvent of the first spinning dope comprises less than 10 vol. % water. According to some embodiments, said solvent of the first spinning dope comprises less than 5 vol. % water. According to some embodiments, said solvent of the first spinning dope is devoid of water.

According to some embodiments, said solvent comprises 70-100 vol. % formic acid and 0-30 vol. % water. According to some embodiments, a solvent comprising 70-100 vol. % formic acid and 0-30 vol. % water is sufficient in cases wherein a spinnable starch-based layer is requested.

As used herein, a “spinnable” fluid or fiber-forming material is any fluid or material that can be mechanically formed into a cylinder or other long shapes by stretching and then solidifying the liquid or material. This solidification can occur by, for example, cooling, chemical reaction, coalescence, or removal of a solvent. The term spinnable may be used interchangeably throughout this specification.

According to some embodiments, said first spinning dope comprises an aqueous solution or dispersion of starch in a solvent.

According to some embodiments, said first spinning dope comprises at least 5 wt. % starch, at least 6 wt. % starch, at least 7 wt. % starch, at least 8 wt. % starch, at least 9 wt. % starch, at least 10 wt. % starch, at least 11 wt. % starch, at least 12 wt. % starch, at least 13 wt. % starch, at least 14 wt. % starch, at least 15 wt. % starch or at least 16 wt. % starch.

According to some embodiments, said first spinning dope comprises at most 50 wt. % starch, at most 45 wt. % starch, at most 40 wt. % starch, at most 39 wt. % starch, at most 38 wt. % starch, at most 37 wt. % starch, at most 36 wt. % starch, at most 35 wt. % starch, at most 34 wt. % starch, at most 33 wt. % starch, at most 32 wt. % starch or at most 31 wt. % starch, at most 30 wt. % starch, at most 29 wt. % starch, at most 28 wt. % starch, at most 27 wt. % starch, at most 26 wt. % starch, at most 25 wt. % starch, at most 24 wt. % starch, at most 23 wt. % starch, at most 22 wt. % starch, at most 21 wt. % starch or at most 20 wt. % starch.

According to some embodiments, said first spinning dope comprises 5-40 wt. % starch. According to some embodiments, said first spinning dope comprises 15-25 wt. % starch. According to some embodiments, said starch is high-amylose starch. According to some embodiments, said starch has an amylose:amylopectin ratio of 60:40-95:5.

According to some embodiments, said first spinning dope comprises 10-30 wt. % starch in 70-90 vol. % formic acid.

According to some embodiments, the method of the invention (e.g., the electrospinning step) takes place in room temperature. According to some embodiments, said electrospinning step takes place in a temperature in the range of 18-24° C., 19-21° C., or 20-22° C.

In some embodiments, the starch-based fiber may further undergo a starch cross-linking step, using cross-linking element known in the art.

According to some embodiments, said one or more of said spinning dopes comprises cells and/or molecules of interest. According to some embodiments, said first spinning dope comprises cells and/or molecules of interest. According to some embodiments, said second spinning dopes comprises cells and/or molecules of interest.

According to some embodiments, said cell is an animal cell. According to some embodiments, said cell is a mammalian cell. According to some embodiments, said cell is a human cell.

According to some embodiments, said cell is a probiotic microorganism. According to some embodiments, said probiotic microorganism is selected from the group consisting of bacteria, yeast and mold, or combinations thereof.

None limiting examples of probiotic microorganism which may be entrapped and/or encapsulated within one or more fibers of the invention include Arthrobacter, Arcanobacterium, Aureobacterium, Aerococcus, Aspergillus, Bacillus, Brevibacterium, Bifidobacterium, Bacteroides, Corynebacterium, Citrobacter, Clostridium, Debaromyces, Ewingella, Enterobacter, Escherichia, Enterococcus, Fusobacterium, Gardnerella, Hafnia, Kurthia, Klebsiella, Kluyvera, Kocuriaw, Lactococcus, Lactobacillus, Leuconostoc, Leciercia, Leminorella, Microbacterium, Micrococcus, Moellerella, Melissococcus, Oenococcus, Obesumbacterium, Propionibacterium, Pediococcus, Peptostreptococcus, Pseudocatenulatum, Pragia, Pantoea, Photorhabdus, Proteus, Providencia, Rothia, Rahnella, Saccharomyces, Streptococcus, Staphylococcus, Stomatococcus, Serratia, Weissella, and combinations thereof.

None limiting examples of yeast microorganism which may be entrapped and/or encapsulated within one or more fibers of the invention include Saccharomyces, Debaromyces, Candida and Pichia. Non-limiting examples of mold includes Aspergillus, Rhizopus, Mucor, and Penicillium.

None limiting examples of molecule of interests which may be entrapped and/or encapsulated within one or more fibers of the invention include antioxidants, vitamins, minerals, proteins, bioactives, phytonutrients and combinations thereof.

None limiting examples of molecule of interests which may be entrapped and/or encapsulated within one or more fibers of the invention include antioxidants selected from the group consisting of flavanoids, cartonoids, tocotrienol, tocopherol, terpenes, coenzyme Q1O, lignan, lycopene, polyphenols, selenium, vitamins and combinations thereof.

Non-limiting examples of vitamins include Vitamins A, B-complex (such as B-1, B-2, B-6 and B-12), C, D, E and K, niacin and acid vitamins such as pantothenic acid and folic acid and biotin. Non-limiting examples of minerals include calcium, iron, zinc, magnesium, iodine, copper, phosphorus, manganese, potassium, chromium, molybdenum, selenium, nickel, tin, silicon, vanadium and boron. Non-limiting examples of proteins include peptides, free amino acids, and mixtures of amino acids or a combination thereof.

As used herein, non-limiting examples of phytonutrients include those that are flavonoids and allied phenolic and polyphenolic compounds, terpenoids such as carotenoids, and alkaloids; including curcumin, limonin, and quercetin and combinations thereof.

As used herein the term “antioxidant” is preferably understood to include any one or more of various substances (as beta-carotene (a vitamin A precursor), vitamin C, vitamin E, and selenium) that inhibit oxidation or reactions promoted by Reactive Oxygen Species (ROS) and other radical and non-radical species. Additionally, antioxidants are molecules capable of slowing or preventing the oxidation of other molecules. Non-limiting examples of antioxidants include carotenoids, coenzyme Q1O (“CoQ1O”), flavonoids, glutathione Goji (Wolfberry), hesperidine, Lactowolfberry, lignan, lutein, lycopene, polyphenols, selenium, vitamin A, vitamin Bl, vitamin B6, vitamin B 12, vitamin C, vitamin D, vitamin E, and combinations thereof.

None-limiting examples of molecule of interests which may be entrapped and/or encapsulated within one or more fibers of the invention include a drug, a food ingredient, a flavoring agent, a dye, an enzyme, an agricultural agent, a pesticide, an industrial agent, a deodorant, a corrosion inhibitor, a fluorescent dye, a catalyst; and combination thereof.

According to some embodiments, there is provided a composition comprising the fiber of the invention and a carrier.

Additionally, the electrospun fibers of the invention can contain conventional carriers such as plasticizers, pigments, colorants, glidants, stabilization agents, pore formers and surfactants. Optional pharmaceutically acceptable excipients present in the electrospun fibers of the invention include, but are not limited to, diluents, binders, lubricants, disintegrants, colorants, stabilizers, and surfactants.

According to some embodiments, the electrospun fibers of the invention are processed using pharmaceutically acceptable plasticizers. Pharmaceutically acceptable plasticizers are known to one skilled in the art and have been described for instance in Eva Snejdrova and Milan Dittrich (2012), Pharmaceutically Used Plasticizers, Recent Advances in Plasticizers, Dr. Mohammad Luqman (Ed.), ISBN: 978-953-51-0363-9. None limiting examples of hydrophilic plasticizers include glycerin, polyethylene glycols, polyethylene glycol monomethyl ether, propylene glycol and sorbitol sorbitan solution. None limiting examples of hydrophobic plasticizers include acetyl tributyl citrate, zcetyl triethyl citrate, castor oil, diacetylated monoglycerides, dibutyl sebacate, diethyl phthalate, triacetin, tributyl citrate, tributyl citrate, triethyl acetyl citrate, and triethyl citrate. Additional plasticizers include dimethyl phthalate, diethyl phthalate, dibutyl phthalate, dibutyl sebacate, triethyl citrate, castor oil and acetylated monoglycerides.

Surfactants can be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenyl ether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine. Non-ionic surfactant can be classified as polyol esters, polyoxyethylene esters, poloxamers. polyol esters includes glycol and glycerol esters and sorbitan derivatives. Fatty acid esters of sorbitan (generally referred to as Spans) and their ethoxylated derivatives (generally referred to as Tweens) are perhaps one of the most commonly used non-ionics.

According to some embodiments, said composition is an edible composition. According to some embodiments, said composition is for oral administration of viable and physiologically active microorganisms and/or at least one compound of interest to an individual in need thereof.

In another embodiment, the shell of the fiber of the invention is substantially devoid of pores (e.g., devoid of pores which enable the efflux of internal core solution through the shell). In another embodiment, said shell is devoid of pores larger than 0.1 nm.

In another embodiment, said composition is for colon delivery. In another embodiment, said shell is decomposed by contact with a stimulus external to the fiber. In another embodiment, said stimulus external to the fiber is a carbohydrase. In another embodiment, said carbohydrase is amylase or maltase. In another embodiment, said decomposed shell allows migration of the core therethrough (e.g., to the colon of a subject).

In another embodiment, said core is a liquid core. In another embodiment, said liquid core comprises a physiological medium suitable for maintaining viability of a microorganism. In another embodiment, said physiological medium comprises sugar (e.g., trehalose).

### Electrospun Fibers

The term “electrospun” or “(electro)sprayed” when used in reference to polymers are recognized by persons of ordinary skill in the art and includes fibers produced by the respective processes. Such processes are described in more detail infra.

Methods for manufacturing electrospun elements as well as encapsulating or attaching cells and molecules thereto are disclosed, inter alia, in WO 2014/006621, WO 2013/172788, WO 2012/014205, WO 2009/150644, WO 2009/104176, WO 2009/104175, WO 2008/093341 and WO 2008/041183.

Manufacturing of electrospun elements can be done by an electrospinning process which is well known in the art. Following is a non-limiting description of an electrospinning process. One or more liquefied polymers (i.e., a polymer in a liquid form such as a melted or dissolved polymer) are dispensed from a dispenser within an electrostatic field in a direction of a rotating collector. The dispenser can be, for example, a syringe with a metal needle or a bath provided with one or more capillary apertures from which the liquefied polymer(s) can be extruded, e.g., under the action of hydrostatic pressure, mechanical pressure, air pressure and high voltage.

The rotating collector (e.g., a drum) serves for collecting the electrospun element thereupon. Typically, but not obligatorily, the collector has a cylindrical shape. The dispenser (e.g., a syringe with metallic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Inverse electrostatic configurations for establishing motions of negatively charged jet from the dispenser to the collector are also contemplated.

At a critical voltage, the charge repulsion begins to overcome the surface tension of the liquid drop. The charged jets depart from the dispenser and travel within the electrostatic field towards the collector. Moving with high velocity in the inter-electrode space, the jet stretches and solvent therein evaporates, thus forming fibers which are collected on the collector, thus forming the electrospun element.

As used herein, the phrase “electrospun element” refers to an element of any shape including, without limitation, a planar shape and a tubular shape, made of one or more non-woven polymer fiber(s), produced by a process of electrospinning. When the electrospun element is made of a single fiber, the fiber is folded thereupon, hence can be viewed as a plurality of connected fibers. It is to be understood that a more detailed reference to a plurality of fibers is not intended to limit the scope of the present invention to such particular case. Thus, unless otherwise defined, any reference herein to a “plurality of fibers” applies also to a single fiber and vice versa. In some embodiments, the electrospun element is an electrospun fiber, such as electrospun nanofiber. As used herein the phrase “electrospun fiber” relates to a fibers formed by the process of electro spinning.

In some embodiments, the electrospun fibers of the invention comprise an electrospun shell and a core. As used herein, the phrase “electrospun shell” refers to an element of a tubular shape, made of one or more polymers, produced by the process of electrospinning. As used herein the phrase “core” refers to an internal layer within the electrospun shell, which comprises a microorganism and/or one or more nutrient(s) and optimally a physiological medium for the viability of said microorganism. In some embodiments, the core is an electrospun core, i.e., prepared by the process of electrospinning. In some embodiments, the microfiber's core is not in a solid state. In some embodiments, the microfiber's core comprises liquid (e.g., medium suitable for cell growth). In some embodiments wherein the microfiber's core is a liquid, said shell has low porosity, as such as to prevent substantial diffusion or leakage of the liquid core.

One of ordinary skill in the art will know how to distinguish an electrospun object from objects made by means which do not comprise electrospinning by the high orientation of the macromolecules, the fiber's morphology (e.g., shell-core), and the typical dimensions of the fibers which are unique to electrospinning.

According to some embodiments of the invention the thickness of the electrospun shell can vary from a few nanometers to several micrometers, such as from about 100 nm to about 20 μm, e.g., from about 200 nm to about 10 μm, from about 100 nm to about 5 μm, from about 100 nm to about 1 μm, e.g., about 500 nm. In another embodiment, the composition comprises a core having a diameter in the range of about 50 nm to about 1 micrometer.

According to some embodiments of the invention, the electrospun fiber is produced by a method which comprises co-electrospinning two or more solutions through multi-axial capillaries, wherein a first solution of the two solutions is for forming an exterior layer of the fiber (e.g., a shell), a second solution of the two or more solutions is for forming a layer external to said exterior layer (e.g., a core within the shell) and so on.

As used herein the phrase “co-electrospinning” refers to a process in which at least two solutions are electrospun from co-axial capillaries (i.e., at least two capillary dispensers wherein one capillary is placed within the other capillary while sharing a co-axial orientation) forming the spinneret within an electrostatic field in a direction of a collector. The capillary can be, for example, a syringe with a metal needle or a bath provided with one or more capillary apertures from which the solution can be extruded, e.g., under the action of hydrostatic pressure, mechanical pressure, air pressure and/or high voltage.

For forming a core/shell structure by electro spinning, a first solution is injected into the outer capillary of the co-axial capillaries while a second solution (also referred herein as a core solution) is injected into the inner capillary of the co-axial capillaries. In some embodiments wherein the core is not a liquid core, the first solution (which is for forming the shell/sheath of the microfiber) solidifies faster than the core solution. In some embodiments, the formation of core/shell structure also requires that the solvent of the core solution be incapable of dissolving the first solution. The solidification rates of the first and second solutions are critical for forming a core/shell microfiber. As a non-limiting example of a core/shell microfiber of about 100 μm wherein the core is not a liquid core, the solidification of the first solution can be within about 30 milliseconds (ms) while the solidification of the core polymer, if occurs, can be within about 10-20 seconds. The solidification may be a result of polymerization rate and/or evaporation rate.

According to some embodiments, the first spinning dope is for forming a shell and the additional spinning dope is for forming a coat over an internal surface of said shell. According to some embodiments, said first polymeric solution is selected solidifying faster than said second polymeric solution and a solvent of said second polymeric solution is selected incapable of dissolving said first polymeric solution.

According to some embodiments of the invention, the solvent of the polymeric solution evaporates faster than the solvent of second solution (e.g., the solvent of the first solution exhibits a higher vapor pressure than the solvent of the second solution). In one embodiment, the shell solidifies and the core remains in a liquid form. In one embodiment, the shell solidifies faster than the core. The flow rates of the first and second solutions can determine the microfiber's outer and inner diameter and thickness of shell.

In some embodiments, said first spinning dope, said at least one of said spinning dope, or both are polymeric solutions, wherein at least one of the polymeric solutions is starch. As used herein the phrase “polymeric solution” refers to a soluble polymer, i.e., a liquid medium containing one or more polymers, co-polymers or blends of polymers dissolved in a solvent. The polymer used by the invention is in preferable embodiments a natural, biocompatible and/or biodegradable polymer.

In some embodiments, at least one of said two or more solutions is devoid of a polymer, such as that the solution is not a spinnable solution so as to for a liquid layer within the fiber. In one embodiment, the liquid layer is formed by the first spinning dope. In one embodiment, the liquid layer comprises starch. In one embodiment, the liquid layer is formed by the one or more additional spinning dopes.

Laboratory equipment for electrospinning can include, for example, a spinneret (e.g. a syringe needle) connected to a high-voltage (5 to 50 kV) direct current power supply, a syringe pump, and a grounded collector. A solution such as a polymer solution, sol-gel, particulate suspension or melt is loaded into the syringe and this liquid is extruded from the needle tip at a constant rate (e.g. by a syringe pump).

In some embodiments, parameters of the electrospinning process may affect the resultant substrate (e.g. the thickness, porosity, etc.). Such parameters may include, for example, molecular weight, molecular weight distribution and architecture (branched, linear etc.) of the polymer, solution properties (viscosity, conductivity & and surface tension), electric potential, flow rate, concentration, distance between the capillary and collection screen, ambient parameters (temperature, humidity and air velocity in the chamber) and the motion and speed of the grounded collector. Accordingly, in some embodiments, the method of producing a substrate as described herein includes adjusting one or more of these parameters.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Materials and Methods

Materials.

High-amylose HYLON® VII maize starch (HS) was obtained from National Starch & Chemical, U.K. Moisture content is 15% max and amylose content is 70%, as given by the producer. Formic acid (FA) (98% purity) was provided from Sigma. All materials were used as received, without further purification.

Spectroscopy.

FTIR spectroscopy was done on Nicolet 380, Thermo Scientific FTIR spectrometer equipped with ATR diamond accessory. Spectra of starch-based thin film and electrospun fibers were obtained by accumulation of 32 scans from 4000 to 650 cm⁻¹ in transmission mode.

DLS.

Dynamic light scattering analysis was performed on Zetasizer Nano Series, Malvern Instruments Ltd. Solutions (1 wt. %) of HS in different concentrations of FA were prepared a day before the analysis.

Viscosity Measurements.

A Discovery DHR-2 rotational rheometer (TA Instruments, USA) was used for investigation of rheological properties of solutions under steady-state shear flow. Parallel-plates geometry with a diameter of 40 mm and a gap value of 0.3 mm was applied for concentrated solutions. Double gap concentric cylinders geometry with the bob diameter of 35 mm and the gaps of 1 mm was used for testing of diluted solutions. For the estimation of critical entanglement concentration (c*), all starch suspensions in pure, 90 and 80 vol. % FA were previously stirred for 24 h at room temperature to enable complete esterification of starch and solution stability. For the frequency sweep measurements, the frequency (w) was initially fixed at 300 rad/s in order to determine the linear viscoelastic region of the samples investigated. Frequency sweep measurements (0.1-1000 rad/s) were then performed at room temperature.

Electrospinning.

Different vol. % solutions of formic acid were used in this study with HS to obtain the final concentration of 17 wt. %. Solution preparation and electrospinning process were carried out at ambient conditions of temperature and humidity (ranging from 40 to 60%). Polymer solution was put in a plastic syringe with G23 blunt needle and electrospun at 11 cm distance from a collector under flow rate of 0.3 mL/h and high voltage of 17 kV. For aligning the fibers, a rotating disc with diameter of 8 cm, and rotating at the speed of 3400 rpm was used as a collector.

Electron Microscopy.

Samples of electrospun fibers were coated with gold using Emitech sputter coater for 15 s and observed with HM SEM at 0.08 Torr, an electron acceleration voltage of 10 kV and at a working distance of 7.5 mm. For transmission electron microscopy (TEM) purposes, electrospun fibers were deposited on carbon-coated copper grids, coated with carbon in a thin layer, and observed at 300 kV using TEM-Titan microscope.

Mechanical Analysis.

Mechanical properties of electrospun mats were investigated using Q800 Dynamic Mechanical Analyzer (TA Instruments, USA) at room temperature. Samples were about 20 mm long, 5 mm wide and 0.15-0.25 mm thick. Stress-strain curves were obtained at stretching rate of 1% per minute.

### Example 1: Esterification of high-amylose starch in formic acid

Qualitative analysis of esterification of starch in formic acid was performed with FTIR spectrometer. Starch films were casted from 10 wt. % of starch in formic acid and DMSO. Dry films were observed with FTIR-ATR spectrometer and spectra are presented in FIG. 2. DMSO is a good solvent for both amylose and amylopectin components of starch. Unlike formic acid, DMSO does not chemically interact with starch, and herein, it served for a comparison with starch cast film obtained from formic acid. FIG. 2 shows the FTIR spectra of starch cast films from DMSO (solid line) and formic acid (dotted line). Both spectra show O—H stretching vibration from 3000 to 3500 cm⁻¹ and C—H stretching vibrations of the glucose units. While the peak at 6=1652 cm⁻¹ reflects the —C—O— stretching vibrations for native starch in DMSO (FIG. 2, solid line), cast film of starch in formic acid showed additional peak at 1716 cm⁻¹ for —C═O ester stretching vibrations, suggesting the reaction of esterification and formation of starch-formate did take place (FIG. 2, dotted line).

NMR analyses confirmed the chemical transformation of starch in pure formic acid at room temperature, and the degree of substitution was estimated to be 1.3. Considering the low temperatures at which the reaction took place, it was speculated that it is mainly amylose that was chemically transformed to amylose-formate, while amylopectin, less mobile (due to the long branches) and reactive (due to inaccessibility of C6 hydroxyl groups), was not chemically transformed.

### Example 2: Gelatinization of Starch in FA

### Dynamic Light Scattering Analysis

Diluted dispersions of starch in FA/H₂O mixtures (1 wt. %) were analyzed with DLS. Considering the fact that the formic acid is not only a solvent but also a reagent for the starch in the reaction of esterification, starch dispersions were prepared in advance, to make sure the entire polymer reacted in the medium and the equilibrium state is reached before the analysis. Thus, starch-formate particles in formic acid, as a final product of starch esterification, were analyzed after 24 h of dissolution time. Stable dispersions were observed in the mixtures where FA was a predominant solvent. In the dominantly aqueous solutions of FA at room temperature a partial dissolution took place, while the rest of the polymer precipitated at the bottom of the vial. Therefore, only the solutions of starch-formate in FA as a predominant solvent (60 to 100 vol. %) were discussed herein.

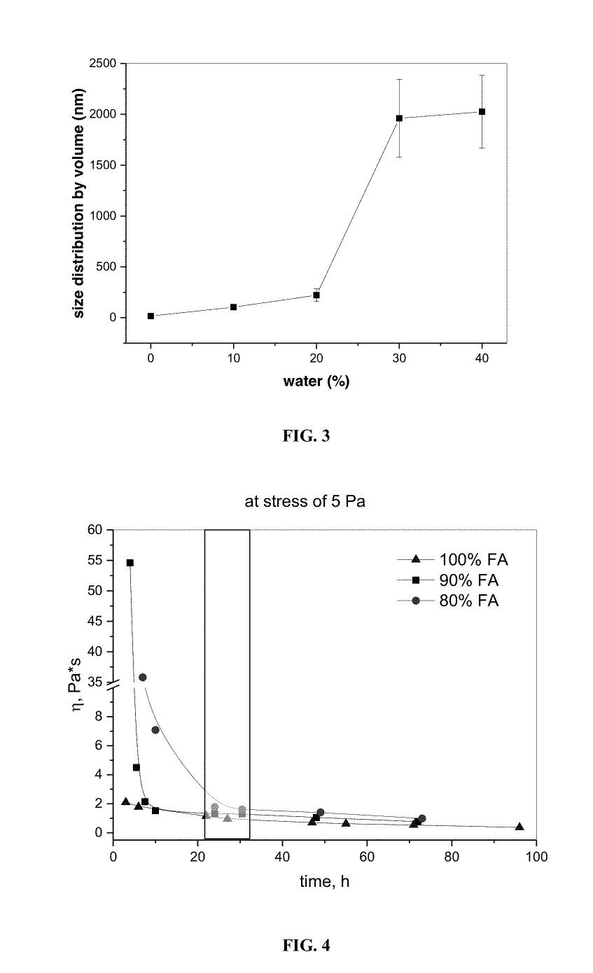

FIG. 3 shows the correlation between the volume-average size distributions of the starch-formate in different FA/H₂O compositions (from pure to 60 vol. % FA). In pure FA, volume average particle size was 17 nm, suggesting the predominant presence of individual coils of amylose and amylopectin in the solution. The presence of individual starch-formate coils in pure FA shows that pure FA has an ability to rapidly and effectively destroy the granule structure even at ambient temperatures. In diluted formic acid systems, however, the presence of water significantly decreased the power of starch swelling and dissolution, while inducing the aggregation of starch, even in dilute solutions. The solutions of 90 vol. % FA had objects of the size of 104 nm, while the particles' size in 80 vol. % FA was of 222 nm (FIG. 3).

Further increase of water content in the solution gave rise to an abrupt increase in the size of the particles. For dispersions of starch-formate in 70 vol. % FA, volume-average size of the particles was ˜1950 nm with very wide size distribution (FIG. 3). Comparable values of particles' size were obtained for 60 vol. % FA solutions (˜2050 nm). The particles measured are still smaller than the average granular size of native high-amylose starch (5-25 μm), indicating to a decreasing impact of formic acid on starch destructuration with the water content increase. High standard-deviation error bars of 70 and 60 vol. % FA starch dispersions, noted in these measurements (FIG. 3), are indicating to the system's heterogeneity.

Taking into account the regioselectivity of the reaction, and higher mobility of amylose compared to amylopectin, it would be reasonable to assume that amylose would preferably react and dissolve in FA while amylopectin would swell and aggregate in water domains. While increasing the water content in FA/water mixtures, degree of substitution of hydroxyl with formyl groups would decrease, directly influencing the final solubility of starch. As a result, for the same concentration of starch, with the increase in water content, the chain entanglement and network formation in the dissolved fraction will decrease and therefrom the viscoelasticity of the system. The viscosity and viscoelasticity behavior of the concentrated starch dispersions was investigated through rheological measurements.

Rheological Studies

A set of concentrations of starch-formate in pure, 90 and 80 vol. % formic acid was measured and the value of the overlap concentration, c* was found to be in the range of 6 to 8 wt. % which is very similar to the results of starch dispersions in aqueous. For all the samples tested, and for the concentrations above 15 wt. %, starch-formate dispersions showed pseudo-plastic behavior, favorable for electrospinning purposes. Herein, 17 wt. % concentration of starch (c>c*) was chosen for the purposes of rheological investigation and electrospinning.

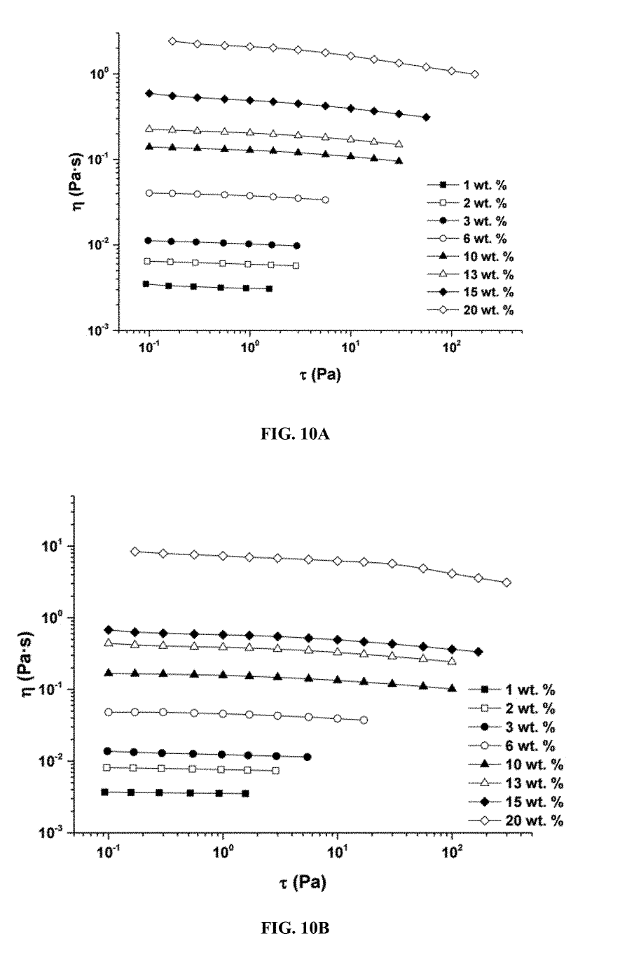

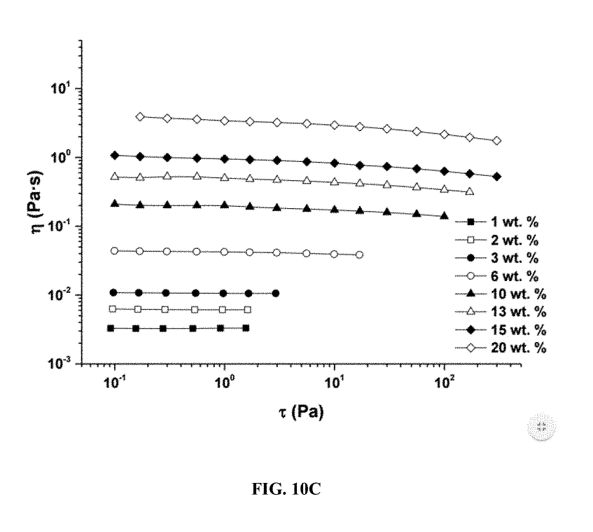

Polymer entanglement was identified as a key factor affecting the transition from the bead morphology, through that of elongated beads or short fibers, to that of continuous fibers. Herein, concentrated systems are not transparent but cloudy or opaque, suggesting some macromolecular aggregation takes place in the solvents used. That is why the appearance of pseudo-plastic behavior, which is typical for entangled polymer solutions, was used as criterion of entanglement network formation (FIGS. 10A-C). It was found that for the concentrations above 10 wt. %, starch-formate dispersions demonstrated pseudo-plastic behavior with a pronounced shear thinning in the region of high shear stresses. However, preliminary attempts of electrospinning showed that the concentration of 17 wt. % was optimal in terms of stability of the process and production of uniform fibers.

In addition, shear viscosity as a function of time was investigated for the same concentration of starch in different formic acid dilutions. FIG. 4 shows viscosity vs. time curves of 17 wt. % starch-formate dispersions in pure (triangles), 90 vol. % (squares) and 80 vol. % formic acid solution (circles). Two distinctive kinetics of the solution were observed: i) fast viscosity decrease, resulting from the reaction of o-formylation and simultaneous dissolution of starch-formate in formic acid, and ii) small changes in viscosity, most likely due to the macromolecular reorganization, and separation between formylated and unreacted fractions of starch. It can be observed that, by changing the water content, the onset time where the kinetics of the system changes dramatically. While in pure formic acid o-formylation and dissolution of starch seem to happen instantly, by increasing the water content in starch/FA dispersions, the time needed for complete swelling and dissolution is prolonged significantly.

Previous studies of structure and crystallinity of starch demonstrated that the amorphous amylose may exist as single helices in a statistical random conformation. The viscosity of single-helix amylose chains would be higher than the viscosity of completely amorphous amylose macromolecules. Therefore, the transition of single-helix amylose chains to statistical Gaussian-like coils lay behind the observed decrease of viscosity of concentrated starch suspensions under stirring.

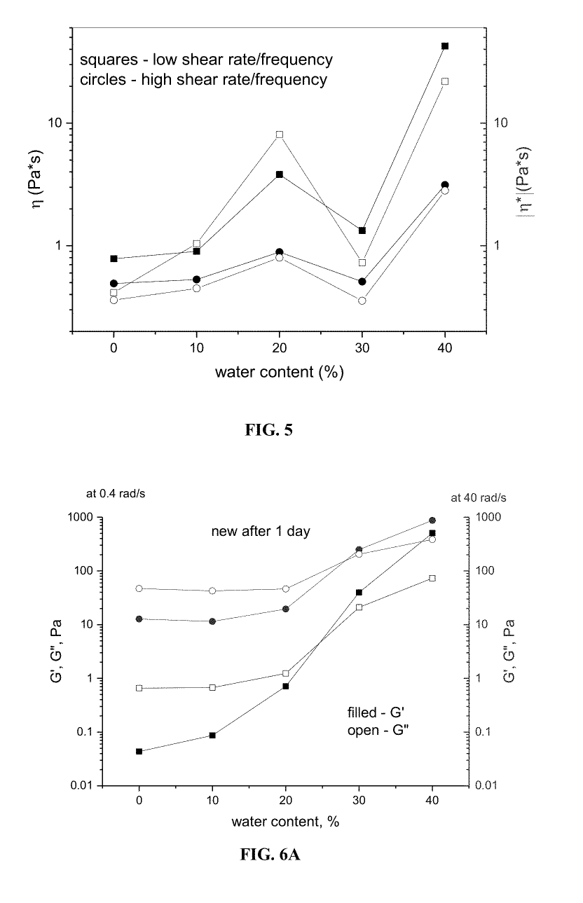

Further investigated was the influence macromolecular structure and particles' size of starch on rheological properties of the system at dynamic and steady state. FIG. 5 represents the viscosity and complex viscosity dependence on water content for different shear rates and frequencies applied, respectively. Dynamic measurements are oscillatory measurements that do not perturb the structure and organization of the system, and show the rheological behavior of starch-FA system at rest. Steady state measurements on the other hand reflect the behavior of the starch-FA system under strong shear forces, similar to those exerted during the electrospinning process under high electric field.

At low frequencies, and for the water content up to 20 vol. %, the complex viscosity increases with the increase of water. In pure formic acid, where starch macromolecules show Gaussian-like organization viscosity is the lowest. With the increase in water content, solubility of starch decreases with the particle size increase (˜100 nm and ˜200 nm for, respectively, 10 and 20 vol. % water in solution). However, if there is enough amount of the dissolved polymer fraction to entangle and form a network connecting swelled particles, the viscosity increase would reflect the cumulative influence of polymer network and increasing size of swelled particles in the dispersion. Further rise in the water content (30 vol. % water) showed large aggregates and cluster formation (particle size of ˜2 μm), and an abrupt decrease in viscosity. The viscosity decrease in this case reflects the weakening of the entanglement forces in the solution. Indeed, for the same starch concentration, if the size of the clusters increases, the fraction of dissolved polymer will decrease as well as the number of chain entanglement points between the polymers, causing the weakening and/or absence of network formation and viscosity decrease. However, 40 vol. % starch dispersions show significant and an abrupt viscosity increase, which could be explained by the fact that in this point of time, the solution behaved like a strong gel. For about the same size of the particles, 30% of water caused the formation of weak gels while 40% showed strong gel formation.

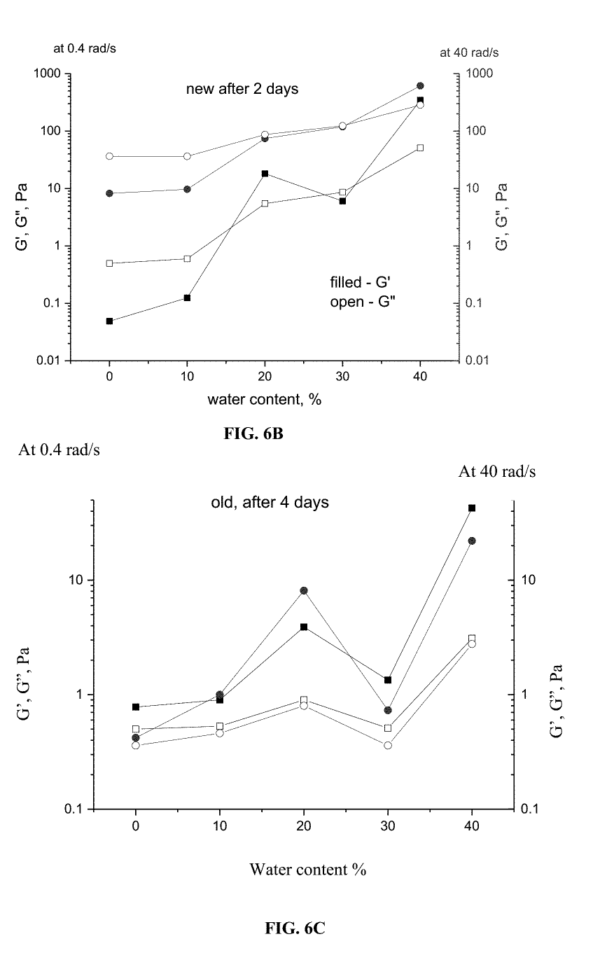

FIG. 6 shows frequency sweep measurements made on samples of 17 wt. % starch in different FA dilutions: from pure to 60 vol. % FA at different periods of time: after 1 day (FIG. 6A), 2 days (FIG. 6B) and 4 days (FIG. 6C). After 1 day, solutions of starch in pure, 90 and 80 vol. % formic acid showed at both high and low frequencies the viscous behavior with G″>G′. On the other hand, 70 and 60 vol. % FA solutions of starch had elastic G′ modulus greater than the viscous G″, which is typical for a gel. By aging, after 2 days (FIG. 6B), starch dispersions in pure and 90 vol. % FA were still showing viscous-like behavior with a slight decrease in absolute values of G′ and G″. Starch in 80 vol. % FA had G′˜G″ indicating to some weak structure formation, while starch dispersions in 70 and 60 vol. % FA had still gel-like behavior. After 4 days of storage, all the solutions demonstrated the retrogradation-like behavior, G′ greater than G″, with the loss of structuration at high frequencies, meaning that the newly formed gel-like structure of the starch in FA is weaker than after 1 or 2 days (FIG. 6A, B) where high frequencies did not manage to break it.

Apparently, in day 1 and 2, double helices of starch (amylose and amylopectin) still constitute in the solution hold by hydrogen bonds between the starch-formate and formic acid and water and physical entanglements of these two polymers. Later, over time, formic acid completely destructurates the double helices structure, amylose and amylopectin are separated (fractionated) and only physical entanglements are present, resulting in weaker behavior of these new gels. The overall higher values of viscosity of starch in 70 and 60 vol. % could be explained by the fact that the starch is only partially swelled in this system and the micron-sized particles are strongly influencing the final viscosity. Additionally, it is known that the water (more significantly present in these systems) is stronger bonded with starch via hydrogen bonding than formic acid, and therefore resulting in greater values of viscosity than the starch solutions with lower water content.

### Example 3: Electrospinning of HS/FA Dispersions

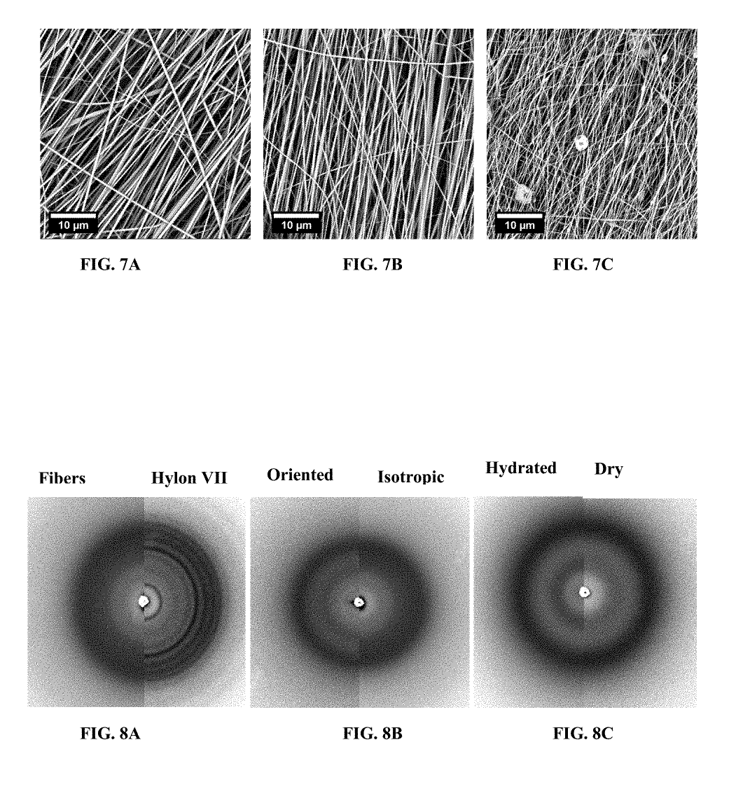

According to the rheological measurements performed previously, 17 wt. % concentration of starch in formic acid was chosen for the electrospinning purposes. Different solvent mixtures of formic acid and water were used for dissolution of starch and the images of resulting electrospun fibers are shown in FIG. 7. Electrospun starch fibers were named hereafter as HS17-pFA, HS17-FA90 and HS17-FA80 obtained from the dispersions of 17 wt. % starch in pure, 90 vol. % and 80 vol. % formic acid, respectively.

As it can be seen from FIG. 7, starch dispersions in pure and 90 vol. % formic gave uniform nanofibers (FIGS. 7 A and B), while 80 vol. % dispersion of starch in formic acid gave beaded fibers (FIG. 7C). Below this solvent composition (70 vol. % and lower), viscous polymer solutions of starch in formic acid did not result in electrospinning and fiber formation (results not shown). This is in accordance with the rheological results presented above, where viscous behavior of the starch dispersions shown in pure, 90 and 80 vol. % FA resulted in electrospun fibers, while starch dispersion in 70 vol. % FA was more gel-like structured and therefore inconvenient for the electrospinning process.

All electrospun fibers were of nanometer size that was decreasing progressively with the increase of water content in formic acid (see Table 1). For HS17-pFA fibers, average diameter was of about 300 nm, while for the HS17-FA90 fibers, the diameter decreased to ˜150 nm. Further decrease in formic acid content in the solution resulted in the fibers having diameter of 84 nm with micron-sized beads (HS17-FA80). Finally, electrospinning of starch dispersion in 70 vol. % FA was non-continuous process of drop formation ending with a fibrous electrospun tale.

TABLE 1
Electrospinning parameters and mean fiber diameters measured for
HS-pFA, HS-FA90 and HS-FA80 fibers, and the zero-shear viscosities
of the electrospinning solutions

| | Formic | | | | | |
|---|---|---|---|---|---|---|
| | acid | Distance | Flow rate | Voltage | Diameter | |
| | (vol. %) | (cm) | (mL h⁻¹) | (kV) | (nm) | η₀ (Pas) |
| HS17- | 100 | 11 | 0.3 | 17 | 304 ± 53 | 1.18 |
| pFA | | | | | | |
| HS17- | 90 | 11 | 0.3 | 17 | 156 ± 33 | 1.31 |
| FA90 | | | | | | |
| HS17- | 80 | 11 | 0.3 | 17 | 84 ± 21 | 1.59 |
| FA80 | | | | | | |

These observations demonstrate a strong influence of water content on starch dissolution, granular-destructuration and consequently processing possibilities of the starch dispersions in formic acid. With the increase of the water content in solution, the quality of the fibers deteriorates dramatically—from uniform fibers obtained from starch in pure FA (and up to 10 vol. % of water), over beaded fibers (20 vol. % of water) to complete electrospray when the water is equal and above 30 vol. %. When compared with the results of DLS, it can be noted that dispersions of starch in formic acid up to the size of 200 nm can be electrospun into fibers. Further increase of water in the system enlarges radically the size of the aggregates to 2 μm, obstructing the electrospinning process and fiber formation. Processability of starch dispersions is in correlation with their rheological properties observed previously. While increasing the water content, solubility of starch decreases, leaving aggregates and clusters weakly bonded with a small amount of dissolved fraction of starch formate (see FIG. 1C). If the aggregates are of the size of few hundreds of nanometers, bead-on-string formation is obtained in electrospinning. If the aggregates are of micron size, due to the absence of the network formation and therefore elasticity, high shear forces during electrospinning lead to droplets formation instead of continuous jet.

Unlike the report from Xu et al. (Biotechnol. Prog., 25: 178-1795, 2009) the results presented in this study suggest that not only solvent properties, but more importantly, particles' size and the presence of polymer network in the electrospinning solution will determine the final quality of the electrospun fibers. This means that there is a limit of electrospinnability of the system starch-aqueous FA where the absence of the polymer network in the solution due to the poor solubility of starch results in breaking of polymer jet under high electric field and bead formation.

This limit lies at the border where the starch particles' size shifts from nano to micron dimensions (see, FIG. 3). While electrospinnable starch dispersion (pure, 90 and 80 vol. % FA) contains nano-sized particles that are swelled and connected in a network, non-electrospinnable starch dispersions (70 and 60 vol. % FA) are more likely to contain densely packed micron-sized particles without the network formation with dissolved polymer fraction. The particle's size in the dispersion of up to ˜100 nm and 90 vol. % FA can be electrospun into uniform fibers, while larger particle aggregates of ˜200 nm and 80 vol. % FA lead to the formation of beaded fibers. This proves that the network formation between the swelled particles and dissolved polymer is strong enough to resist elongational forces during electrospinning and form fibers. Further increase in water content in the system (70 and 60 vol. % FA), hindered significantly electrospinning process, and resulted in process instability and electrospraying, caused most probably by the presence of micron-sized aggregates and absence of a polymer network and entanglement formation.

Additionally, as rheological studies suggested, starch dispersions in FA are susceptible to age, and for the same starch concentration, different FA/water composition showed different time window for successful electrospinning process. This time window shifted towards longer times with the increase in water content. This is in complete agreement with dynamic rheology tests where after a certain time, specific for the starch-FA system in question, polymer dispersion behaves as a gel and it is therefore no longer suitable for the electrospinning purposes.

### Example 4: Microstructure Study

All previous results suggest complete or partial starch-granule destructuration in pure or aqueous formic acid solutions respectively. To confirm the presence of amorphous starch-formate inside the electrospun fibers resulting from starch-granule destructuration, X-ray diffraction and polarized microscopy measurements and were applied.

FIG. 8 shows WAXS patterns of fibers compared with initial structure of the HYLON® VII starch powder (FIG. 8A), oriented and isotropic (FIG. 8B), as well as hydrated and dry oriented fibers in a capillary (FIG. 8C). FIG. 8A displays a distinctive difference in WAXS patterns for native starch and electrospun starch-formate fibers with the fibrous mat having a typical pattern of an amorphous material. This is a clear evidence for starch destructuration confirming previous observations by DLS and viscosity measurements. Both samples of HS17-pFA and HS17-FA80 showed typically amorphous WAXS patterns. There is a minor difference between the orientated and isotropic mat (FIG. 8B) with cyclical rings becoming slightly more intensified. This suggests to a weak recovery of the molecular organization inside the sample for oriented fibers after hydration in temperature/humidity-controlled chamber for 36 h. More pronounced rings of hydrated fibers might indicate to an increase in the molecular organization inside the sample, but without completely returning to the initial crystalline structure of HYLON® VII powder.

The orientation of the macromolecules and/or micro-domains inside the electrospun fibers and possible crystallinity are expected to be shown through the elliptical and 4-point WAXS patterns as observed at SEBS tri-block copolymer electrospun fibers by Rungswang et al.¹⁷ In our case, micro-domain and molecular orientation were not observed as the rings remained circular—both isotropic, randomly collected and oriented fibers. Seeing only circular and wide rings is suggesting that there is no preferential orientation of the polymer inside the both isotropic and oriented electrospun fibers.

While the isotropic fibers give wide and dark circular rings, aligned fibers show narrower ring signals. However, the overall look to these patterns does not give the impression of the orientation of any kind. A small difference could be observed close to the beam-stop and an elliptical imprint in the case of oriented fiber mat. This orientation indicates a fiber orientation, or orientation of the porous structure between the fibers, but not the presence nor orientation of the crystalline domains within the fibers.

### Example 5: Mechanical Properties of Nanofibers

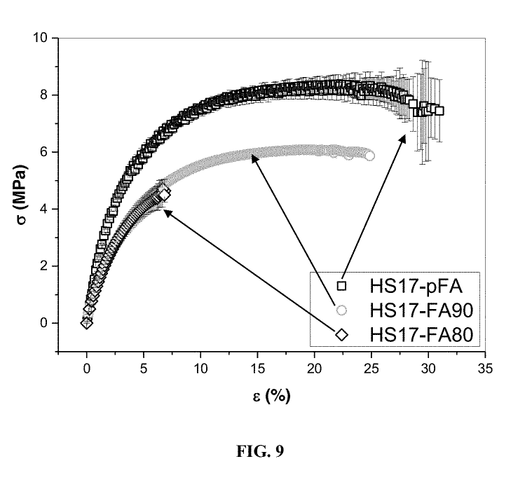

Typical stress-strain curves of electrospun fibers from different FA compositions are shown in FIG. 9. FIG. 9 summarizes mechanical response of the HS17-pFA, HS17-FA90 and HS17-FA80 electrospun fibers.

From the curves presented in FIG. 9, and for each type of fibrous mat, it was extracted: maximum stress (σₘₐₓ), elongation at break (ε*) and Young's modulus (E_{0.5}) (Table 2).

TABLE 2
Mechanical properties of native HYLON VII ®
starch and starch-formate electrospun fibers.

| | Σₘₐₓ, MPa | ε*, % | E_{0.5}, MPa |
|---|---|---|---|
| HS17-pFA | 8.1 ± 1.0 | 26 ± 5.0 | 241 ± 37 |
| HS17-FA90 | 6.1 ± 0.4 | 21 ± 2.0 | 178 ± 9 |
| HS17-FA80 | 4.5 ± 0.5 | 6.7 ± 0.3 | 167 ± 18 |
| HYLON ® VII cast film⁴⁸ | 40 ± 13 | 1.92 ± 1.0 | 3390 ± 387 |

It could be observed the overall trend of a decrease in mechanical properties of the fibers with the increase of water content in the electrospinning solution. While starch-formate fibers electrospun from pure FA solutions gave high values of maximum stress, elongation at break and Young's modulus (σₘₐₓ=8.1 MPa, ε* of 26% and E_{0.5}=241 MPa), these values were significantly decreased by double for σₘₐₓ and E_{0.5} when the value of elongation at break was reduced 4 times for HS17-FA80 electrospun fibers (σₘₐₓ=4.5 MPa, ε* of 6.7% and E_{0.5}=167 MPa).

When compared to the average diameters of the fibers tested, the fibers having the highest diameter (HS17-pFA) had the highest values of maximum stress, elongation at break and Young's modulus. HS17-FA90 fibers electrospun from 90 vol. % formic acid showed slightly lower values for σₘₐₓ, ε* and E_{0.5}, when HS17-FA80 electrospun fibers evidenced decreased values for σₘₐₓ and E_{0.5} by half, and elongation by 4 times lower than for the fibers electrospun from pure formic acid.

Koch et al. (Int. J. Biol. Macromol. 2010, 46, 13-19.) studied mechanical properties of cast starch films from the high-amylose corn starch (HYLON® VII), and they measured Young's modulus of 3390±387 MPa, tensile strength of 40±13 MPa and elongation at break of 1.92±1.0%. Compared to electrospun fibers, it could be observed that the Young's modulus and tensile strength decreased significantly compared to the high-amylose starch films. On the other hand, elongation at break was notably higher for electrospun fibers and decreased towards the value for high-amylose starch film when the formic acid concentration in the dispersion decreased.

The present invention presents for the first time a straight-forward method for processing starch from formic acid (FA) solutions. The dual role of formic acid consisted in simultaneous destructuration of the starch granule-structure, esterification of starch to starch-formate and as dispersing medium for electrospinning process. At ambient temperatures during the solution preparation and electrospinning process, nanofibers with the diameters of about 200 nm were produced. Rheological measurements evidenced complete starch-granule destructuration in pure formic acid solutions at ambient temperatures, while the destructuration was only partial for aqueous dispersions of starch-formate in formic acid. Final fibrous mat showed decreased crystallinity and improved mechanical properties highlighting its potential as economic and ecological biomaterial, ready to be used in food packaging or pharmaceutical industry.

### Example 6: Encapsulation of Lactobacillus Bacteria in High Amylose Corn Starch (HACS)—Formate

Dry live bacteria were encapsulated in HACS-formate to demonstrate the possible application of a probiotic product that will pass the stomach and small intestine intact and be released in the large intestine.

HACS is dissolved in Formic acid (FA), as detailed above, to produce starch-formate that can be electrospun to produce fibers.

Two novel systems for producing hollow HACS-formate fibers (also denoted “electrospun tubes”), were used: the first used glycerol as a core, the later used oils as a core. The glycerol system produces tubes with diameter in the order of 2-10 μm. The oil system produces tubes in the order of 1 μm, and the oil stays inside the tubes.

Bacteria was either freeze-dried or dried using glycerol, as further described. Bacteria was freeze-dried with sugar encapsulation (Italian powder) with large particles up to about 500 μm. The particles can be ground in a mortar (with some loss of viable bacteria) to particles up to about 200 μm. Alternatively, to receive glycerol dried bacteria, individual bacteria or bacterial chains were dried gradually by increasing glycerol concentration slowly.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method of making a starch-formate fiber, the method comprising the steps of:
providing a first spinning dope comprising a solution or dispersion of starch in a solvent comprising at least 50% vol. formic acid, where the starch is present at a concentration above the critical entanglement concentration where starch fibers are to be produced;
electrospinning the spinning dope to produce a starch-formate fiber.

2. A method of making a starch-formate concentric multi-layered fiber, the method comprising the steps of:
providing a first spinning dope for forming at least one layer of the fiber, the first spinning dope comprises a solution or dispersion of starch in a solvent comprising at least 50% vol. formic acid;
providing one or more additional spinning dopes for forming at least one additional layer within said fiber;
co-electrospinning the spinning dopes through multi-axial capillaries to produce a starch-formate concentric multi-layered fiber.

3. The method of claim 1, wherein said solvent of the first spinning dope comprises at least 70% vol. formic acid.

4. The method of claim 1, wherein said solvent of the first spinning dope comprises at least 70% vol. formic acid.

5. (canceled)

6. (canceled)

7. (canceled)

8. The method of claim 1, wherein said first spinning dope comprises 5-40 wt. % starch.

9. (canceled)

10. (canceled)

11. The method of claim 1, wherein said first spinning dope comprises 10-30 wt. % starch in 70-90 vol. % formic acid.

12. (canceled)

13. The method of claim 2, wherein the first spinning dope is for forming a shell and the additional spinning dope is for forming a coat over an internal surface of said shell.

14. The method of claim 1, wherein said one or more of said spinning dopes comprises cells and/or molecules of interest.

15. The method of claim 1, wherein said cell is an animal cell, a probiotic microorganism, bacteria, yeast, mold, or any combination thereof.

16. (canceled)

17. (canceled)

18. A fiber comprising electrospun starch-formate, said starch has an amylose:amylopectin ratio of 60:40-95:5.

19. A concentric multi-layered fiber comprising at least one layer comprising starch-formate, said starch has an amylose:amylopectin ratio of 60:40-95:5.

20. The fiber of claim 18 having a diameter of 50-500 nm.

21. A composition comprising the fiber of claim 18 and a carrier.

22. The composition of claim 21 for oral administration of viable and physiologically active microorganisms and/or at least one compound of interest to an individual in need thereof.

23. The method of claim 2, wherein said first spinning dope comprises 10-30 wt. % starch in 70-90 vol. % formic acid.

24. The method of claim 2, wherein said one or more of said spinning dopes comprises cells and/or molecules of interest.

25. The method of claim 2, wherein said cell is an animal cell, a probiotic microorganism, bacteria, yeast, mold, or any combination thereof.

26. The fiber of claim 19 having a diameter of 50-500 nm.

27. A composition comprising the fiber of claim 19 and a carrier.
